(19) United States
(12) Patent Application Publication
(10) Pub. No.: US 2020/0200900 A1
(43) Pub. Date: Jun. 25, 2020

(54) Ultrasonic Imaging Device and Control Method Thereof

(71) Applicant: Hitachi, Ltd., Tokyo (JP)

(72) Inventors: Rei ASAMI, Tokyo (JP); Nobuhiko FUJII, Tokyo (JP); Hiroki TANAKA, Tokyo (JP)

(21) Appl. No.: 16/680,669

(22) Filed: Nov. 12, 2019

(30) Foreign Application Priority Data
Dec. 19, 2018 (JP) 2018-237370

(51) Int. Cl. G01S 15/89; A61B 8/06; G01S 7/52; G01S 15/58; A61B 8/00; A61B 8/08

(52) U.S. Cl. CPC G01S 15/8988; A61B 8/06; G01S 7/52053; G01S 15/584; G01S 7/5202; A61B 8/488; A61B 8/5246; G01S 7/52034; A61B 8/461

(57) ABSTRACT

An object of the invention is to provide an ultrasonic imaging device that has a function of setting an optimal velocity range and baseline as initial settings for a target blood flow at the start of spectral Doppler. A control unit that controls transmission and reception of an ultrasonic imaging device performs a first blood flow measurement for acquiring a two-dimensional distribution of blood flow information, and a second blood flow measurement for acquiring a spectrum of a blood flow velocity. A calculation unit that performs Doppler calculation includes: a blood flow velocity estimation unit that estimates a blood flow velocity causing no aliasing, and a measurement condition calculation unit that calculates a measurement condition of the second blood flow measurement by using the blood flow velocity causing no aliasing. The control unit starts the second blood flow measurement under the measurement condition calculated by the measurement condition calculation unit.

### TECHNICAL FIELD

The present invention relates to an ultrasonic imaging device, and particularly to a technique of automatically adjusting a velocity range or the like in a blood flow measurement using the ultrasonic imaging device.

### BACKGROUND ART

The blood flow measurement using an ultrasonic imaging device is roughly divided into Doppler imaging such as a color Doppler method and a power Doppler method, and a spectral Doppler method such as a pulse Doppler method and a continuous Doppler method. Doppler imaging is a method of visualizing a blood flow by two-dimensionally displaying a Doppler signal received by an ultrasonic probe, and in the spectral Doppler method, velocities obtained by performing frequency analysis on a Doppler signal are displayed in a spectrum.

The spectral Doppler method is used to measure a blood flow change at a certain single point in the body. Therefore, generally, a region including a measurement target is first imaged by Doppler imaging, and then a user determines the measurement target based on information obtained by Doppler imaging. In addition, spectral Doppler is started. At this time, measurement conditions such as a velocity range, a baseline, and a blood flow direction are adjusted to optimize a displayed spectrum according to the blood flow. Particularly, the adjustment of the velocity range is a necessary operation, when the velocity range is too wide for a target blood flow, the spectrum is vertically compressed and the velocity resolution is reduced. When the velocity range is too narrow, aliasing occurs in the spectrum, which makes it difficult to determine the velocity.

With respect to determination of a spectral Doppler measurement position of Doppler imaging, PTL 1 discloses a method of automatically setting a spectral Doppler measurement point, by obtaining a high-speed blood flow part based on information obtained by Doppler imaging. However, in this technique, settings of the velocity range and the like necessary for spectral Doppler are not performed.

Meanwhile, a method has been proposed to automate the adjustment of the velocity range and the like. For example, PTL 2 discloses a method of creating a histogram of velocity components based on spectral images, and performing optimization based on a spectral image in which a velocity component of the maximum frequency in the histogram is present.

### PRIOR ART LITERATURE

### Patent Literature

PTL 1: JP-A-2009-22463

PTL 2: Japanese Patent No. 5443082

### SUMMARY OF INVENTION

### Technical Problem

The method described in PTL 2 has an effect of making it possible to avoid complicated processing that is performed manually in the related art, by automating the adjustment of the velocity range and the like. However, since it is necessary to acquire spectral images in order to realize automated adjustment in this method, it takes time to complete the adjustment after a spectral Doppler measurement is started. Generally, since a blood flow velocity is affected by pulsation, it is necessary to measure one or more heartbeats in order to obtain a histogram of the velocity, and it is necessary to wait for at least one second or more.

Further, in the method described in PTL 2, an appropriate histogram may not be obtained since the aliasing occurs when an initial setting velocity range is too narrow.

An object of the invention is to provide an ultrasonic imaging device that has a function of setting an optimal velocity range and baseline as initial settings for a target blood flow at the start of spectral Doppler.

### Solution to Problem

In order to solve the problem describe above, in the invention, information necessary for setting of measurement conditions (velocity range and the like), under which the blood flow velocity does not causes aliasing in a spectral Doppler target measurement position, is collected in Doppler imaging performed before spectral Doppler, and an optimal measurement condition is calculated and automatically set as an initial setting before spectral Doppler is started.

That is, an ultrasonic imaging device of the invention includes: a transmission and reception circuit that transmits and receives an ultrasonic signal via an ultrasonic probe; a calculation unit that performs Doppler calculation using an ultrasonic signal received by the transmission and reception circuit; and a control unit that controls an operation of the transmission and reception circuit, and performs a first blood flow measurement for acquiring a two-dimensional distribution of blood flow information and a second blood flow measurement for acquiring a spectrum of a blood flow velocity. The calculation unit includes a blood flow velocity estimation unit that estimates a blood flow velocity causing no aliasing using an ultrasonic signal acquired before transmission and reception of a ultrasonic wave in the second blood flow measurement is started, and a measurement condition calculation unit that calculates a measurement condition of the second blood flow measurement using the blood flow velocity causing no aliasing.

Further, a control method of an ultrasonic imaging device of the invention is a control method of an ultrasonic imaging device including a transmission and reception circuit that transmits and receives an ultrasonic signal via an ultrasonic probe and a calculation unit that performs Doppler calculation using an ultrasonic signal received by the transmission and reception circuit, the control method includes: a step of causing the transmission and reception circuit to perform a first blood flow measurement for acquiring a two-dimensional distribution of blood flow information and a second blood flow measurement for acquiring a spectrum of a blood flow velocity; and a step of causing the calculation unit to perform calculation of estimating a blood flow velocity causing no aliasing using an ultrasonic signal acquired during the first blood flow measurement and to perform calculation of calculating a measurement condition of the second blood flow measurement using the blood flow velocity causing no aliasing, and transmission and reception of an ultrasonic signal of the second blood flow measurement is started under the measurement condition calculated by the calculation unit.

### Advantageous Effect

According to the invention, an optimal velocity range and baseline for a target blood flow can be set without delay at the start of spectral Doppler.

### BRIEF DESCRIPTION OF DRAWINGS

FIG. 1 is a block diagram showing an overall configuration of an ultrasonic imaging device.

FIG. 2 is a functional block diagram of a measurement condition calculation unit according to a first embodiment.

FIG. 3 is a flowchart showing a flow of operations of the ultrasonic imaging device according to the first embodiment.

FIGS. 4A and 4B are diagrams each showing an example of a UI displayed on a display unit during a color Doppler measurement.

FIG. 5 is a flowchart showing processing of the measurement condition calculation unit according to the first embodiment.

FIGS. 6A to 6C are diagrams showing examples of transmission and reception sequence for avoiding aliasing.

FIG. 7 is a diagram showing a histogram example of a blood flow distribution.

FIGS. 8A and 8B are diagrams showing calculation of a minimum blood flow velocity and a maximum blood flow velocity from the histogram, in which FIG. 8A shows a blood flow velocity distribution before aliasing correction, and FIG. 8B shows a blood flow velocity distribution after aliasing correction.

FIGS. 9A to 9C are diagrams each showing a display example of blood flow information calculated by a blood flow velocity estimation unit.

FIG. 10 is a flowchart showing processing of a measurement condition calculation unit according to a first modification.

FIG. 11 is a functional block diagram of a measurement condition calculation unit according to a second embodiment.

FIG. 12 is a flowchart showing processing according to the second embodiment.

FIG. 13 is a diagram showing examples of a transmission and reception sequence according to a third modification.

FIG. 14 is a diagram showing a measurement position candidate according to the third modification.

### DESCRIPTION OF EMBODIMENTS

Embodiments of an ultrasonic imaging device and an imaging method of the invention will be described with reference to the drawings.

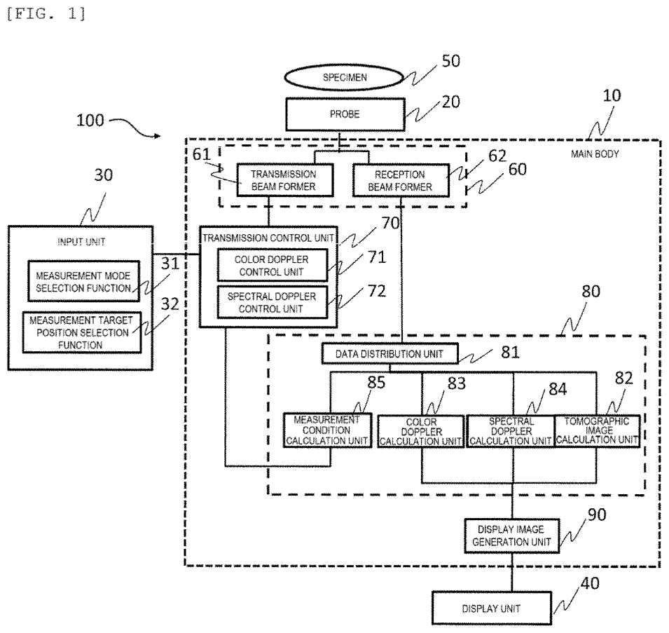

First, an overall configuration of the ultrasonic imaging device common to each of the embodiments will be described with reference to FIG. 1. As shown in FIG. 1, an ultrasonic imaging device 100 includes: a main body 10; an ultrasonic probe 20 that transmits and receives an ultrasonic wave by contacting a specimen 50; an input unit 30 that is used by a user to input a condition or the like necessary for a measurement and control; and a display unit 40 that displays an image or a spectrum that is a measurement result, and a UI.

The main body 10 include: a transmission and reception circuit 60 that is connected to the ultrasonic probe 20; a transmission control unit 70 that controls timing of transmission and reception and the like; a signal processing unit (calculation unit) 80 that performs Doppler calculation and tomographic image calculation using a received signal; and a display image generation unit 90 that generates an image to be displayed on a display device. Although a control unit that controls components of the ultrasonic imaging device 100 in addition to the transmission and reception may be provided, the transmission control unit 70 also functions as a general control unit.

The ultrasonic imaging device according to the present embodiment performs two measurements, one of which is a measurement for visualizing blood flow information as a two-dimensional distribution (color Doppler) and the other of which is a blood flow measurement for displaying blood flow velocities of a predetermined region in a spectrum. For this reason, in addition to a function of setting an imaging condition or a scan condition, the input unit 30 generally includes a function (measurement mode selection unit) 31 of selecting a measurement mode, a function (measurement target selection unit) 32 of selecting a position that is a measurement target in spectral Doppler, and the like. Examples of an imaging method include a planar imaging method of imaging a two-dimensional cross section and a stereoscopic imaging method of imaging a three-dimensional region, or either of which may be adopted. A spectral Doppler scan method may be either a method using a continuous wave or a method using a pulse wave.

The ultrasonic probe 20 is a device in which a plurality of transducers (transducers) are arranged in a one-dimensional direction or a two-dimensional direction, and irradiates the specimen 50 with an electric signal, as an ultrasonic signal, from the transmission and reception circuit 60, and detects an echo signal that is a reflected wave from the specimen 50.

The transmission and reception circuit 60 includes an oscillator that generates a signal of a predetermined frequency, and includes a transmission circuit (ultrasonic transmission unit) that transmits a drive signal to an ultrasonic probe in a predetermined scanning method, and a reception circuit (ultrasonic reception unit) that performs signal processing such as phasing addition, wave detection, and amplification on the echo signal received by the ultrasonic probe. The transmission circuit can include a transmission beam former 61 that gives each transducer of the ultrasonic probe a separate delay time, and gives directivity to an ultrasonic beam; and the reception circuit can include a reception beam former (phasing addition unit) 62 that gives the delay time to a signal received by each transducer and adds up the delay time. The received signal output from the reception circuit after beamforming is a Radio Frequency (RF) signal having a frequency component dependent on the blood flow velocity, and is input to a signal processing unit 80 as data for each frame (frame data). An A/D converter is provided in the reception circuit or downstream of the reception circuit, and the RF signal is input to the signal processing unit 80 as an A/D converted digital signal.

The transmission control unit 70 includes a color Doppler control unit 71 and a spectral Doppler control unit 72, and controls an operation of the transmission and reception circuit 60 so as to perform measurements under the imaging condition and the scan condition respectively, in accordance with a measurement mode received by the input unit 30. In the present embodiment, the transmission and reception circuit 60 is controlled so as to continuously perform the two measurements of color Doppler and spectral Doppler. In addition, the transmission control unit 70 transmits and receives a pulse wave for estimating a velocity at which aliasing does not occur in spectral Doppler, in parallel with normal color Doppler scan (scan of the ultrasonic beam) or in a period (intermediate period) of transitioning from color Doppler to spectral Doppler.

The signal processing unit 80 processes a signal (digital RF signal) received by the reception circuit, creates an ultrasonic tomographic image, and calculates a blood flow velocity. For this reason, the signal processing unit 80 includes calculation units such as a data distribution unit 81 that distributes received signals (frame data) into signals for creating the tomographic image and signals for calculating the blood flow velocity in accordance with the measurement mode, a tomographic image calculation unit 82 that generates a tomographic image such as a B-mode image, a color Doppler calculation unit 83 that calculates two-dimensional information such as a Doppler velocity and performs color mapping, a spectral Doppler calculation unit 84 that calculates the blood flow velocity of the predetermined region to acquire a spectrum, and a measurement condition calculation unit 85 that calculates a measurement condition of a spectral Doppler measurement.

The calculations performed by the tomographic image calculation unit 82, the color Doppler calculation unit 83, and the spectral Doppler calculation unit 84 are similar to those of an ultrasonic imaging device in the related art, and detailed description thereof will be omitted unless otherwise particularly required.

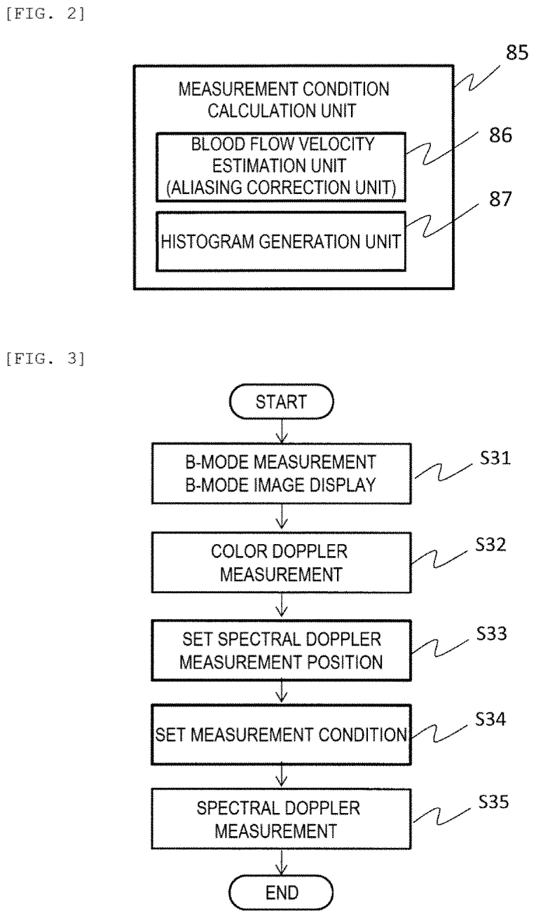

During a measurement being performed under the control of the color Doppler control unit 71, the measurement condition calculation unit 85 estimates a velocity causing no aliasing (blood flow velocity in which aliasing is corrected) automatically or based on an instruction input via the input unit 30, and calculates a velocity range and a baseline position using the estimated velocity causing no aliasing. For this reason, as shown in FIG. 2, the measurement condition calculation unit 85 may include a blood flow velocity estimation unit 86, and may further include a histogram generation unit 87 for calculation of minimum and maximum blood flow velocities in a predetermined period. A function of the blood flow velocity estimation unit 86 may be performed by the color Doppler calculation unit 83.

The display image generation unit 90 converts the data generated by each of the calculation units 82 to 85 into image data to be displayed on the display unit 40, for example, through scan conversion by a scan converter, and generates a display image in combination with data such as imaging conditions and specimen information to be displayed accompanying the image data.

Apart or all of the functions of the signal processing unit (calculation units) and the transmission control unit 70 (control units) can be implemented in a computer that includes a memory and a Central Processing Unit (CPU) or a Graphics Processing Unit (GPU) by the CPU or the like reading and executing a program containing an arithmetic algorithm for each functional unit. A part of the functions of the calculation unit may be implemented by hardware such as an Application Specific Integrated Circuit (ASIC) or a Field-Programmable Gate Array (FPGA).

The display unit 40 may display a GUI or the like that functions as an input unit, in addition to displaying the image generated by the display image generation unit 90. The display unit 40 also displays a set imaging condition, an imaging condition set by default, information serving as a guide for imaging, an image, and the like.

Next, an embodiment of the blood flow measurement using the ultrasonic imaging device will be described.

### First Embodiment

In the present embodiment, in a Doppler mode, a spectral Doppler measurement (second blood flow measurement) is performed following a color Doppler measurement (first blood flow measurement), and initial measurement conditions of the spectral Doppler measurement are calculated and set in an intermediate period of transitioning from the color Doppler measurement to the spectral Doppler measurement. A flow of imaging in the present embodiment is shown in FIG. 3.

When a measurement mode of the Doppler mode is selected via the input unit 30 (measurement mode selection unit 31), the transmission control unit 70 first starts a B-mode measurement, as a measurement for specifying a measurement target (S31). The B-mode measurement is a measurement for acquiring a tomographic image of a specimen. In the transmission and reception circuit 60, a two-dimensional or three-dimensional region is scanned with an ultrasonic pulse for B-mode, an ultrasonic signal reflected from the region is received, and image data indicating intensity of a signal from each position is generated by the tomographic image calculation unit 82. The display image generation unit 90 generates a tomographic image obtained by converting the signal intensity into a luminance value and displays the tomographic image on the display unit 40. The B-mode measurement is performed with at least one frame.

When a user selects a portion such as a blood vessel or a heart as the measurement target via the input unit 30 (measurement region selection unit 32), based on the tomographic image displayed on the display unit 40, the transmission control unit (color Doppler control unit 71) starts the color Doppler measurement (S32). That is, a selected region is scanned at a predetermined frame rate, and a blood flow velocity of the region is measured. In the color Doppler measurement, ultrasonic pulses are transmitted and received a plurality of times at a predetermined repetition frequency for each scan line. The color Doppler calculation unit 83 calculates a Doppler shift amount using a known calculation method such as autocorrelation calculation, with respect to reception signals obtained with the plurality of times of transmission and reception, and calculates the blood flow velocity. Information about the blood flow velocity obtained here is an average value of blood flow velocities of a certain region on one beam line of the ultrasonic pulse, or a blood flow velocity for each sample. The color Doppler calculation unit 83 may further use the reception signals obtained with the plurality of times of transmission and reception to calculate information about power and variance of a blood flow.

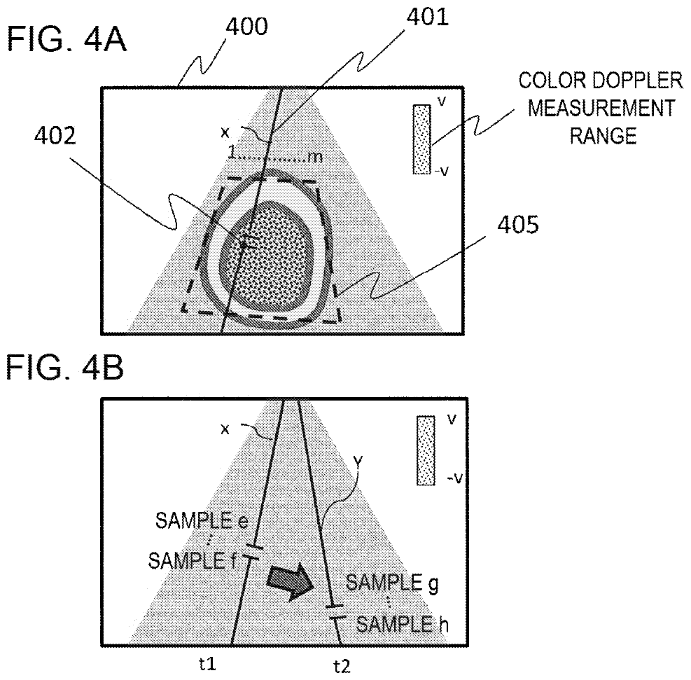

Blood flow information obtained in the color Doppler measurement is superimposed on the tomographic image obtained in the previous B-mode measurement and is displayed on the display unit 40. In this state, when the user inputs an instruction for transition to the spectral Doppler mode (for example, a button of spectral Doppler mode “ON” is operated), as shown in FIG. 4(a), a cursor 401 for selecting a spectral Doppler measurement position is displayed on a screen 400 that displays both a tomographic image of a color Doppler measurement region 405 and a color Doppler measurement range (S33). At this point, the transmission control unit 70 receives the measurement mode transition instruction, but transmission and reception of an ultrasonic pulse for color Doppler is continued while transmission and reception of an ultrasonic pulse for spectral Doppler is not started.

The cursor 401 for selecting the spectral Doppler measurement position is a UI operable by the user, and the user designates an ultrasonic beam direction and a sample window 402 that determines the measurement position by operating the cursor 401 on the screen using a pointing device such as a mouse. In the illustrated example, a sample window of a sample e-a sample f, which are set on a scan line x among a color Doppler scan range of 1 to m, is set. The spectral Doppler measurement position is determined with such an operation of the cursor 401. Next, when an instruction to start the spectral Doppler measurement is input by the user, the transmission and reception of the pulse for spectral Doppler is started at the measurement position (S35).

In a period from selecting the measurement position (S33) to starting the spectral Doppler measurement (S35), that is, in a transition period (intermediate period) in which the transmission and reception of color Doppler is continued while the transmission and reception of the ultrasonic signal for spectral Doppler has not started, the measurement condition calculation unit 85 performs calculation for calculating spectral Doppler measurement conditions (S34). For this reason, first, the blood flow velocity estimation unit 86 performs calculation to estimate a blood flow velocity causing no aliasing, by using the signal acquired while the color Doppler measurement is continued. The measurement condition calculation unit 85 calculates the measurement conditions by using the estimated blood flow velocity causing no aliasing. The measurement conditions include a velocity range and a baseline.

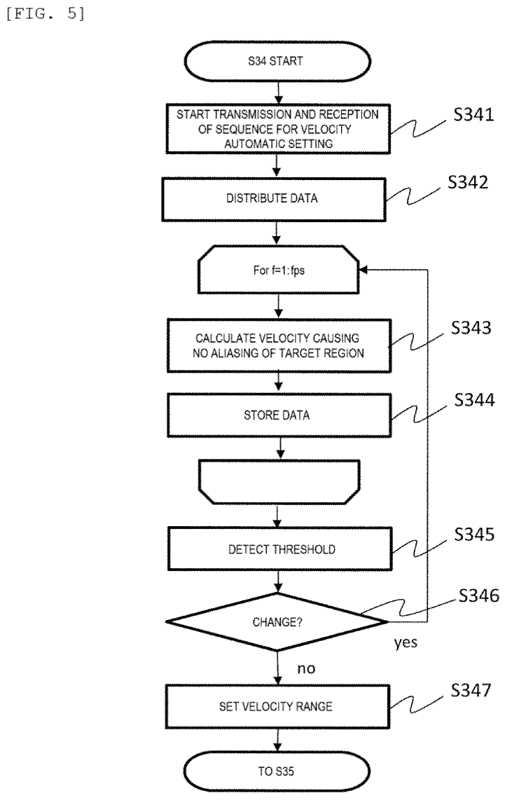

Hereinafter, details of the processing in the intermediate period (S34) will be described with reference to FIG. 5.

The transmission control unit 70 sends a command to the transmission and reception circuit 60, takes the measurement position determined in step S33 or a narrow region including the measurement position (the scan line x and another scan line in the vicinity thereof) as a target, and transmits and receives ultrasonic signals necessary for estimating the blood flow velocity causing no aliasing (S341). With respect to the method of estimating the blood flow velocity causing no aliasing, there are several known methods such as a method of calculating the velocity causing no aliasing by transmitting and receiving a pulse for aliasing avoidance, and a method of calculating the velocity causing no aliasing by correcting a velocity having aliasing, and a case where the method of using the pulse for aliasing avoidance is adopted is described in the present embodiment.

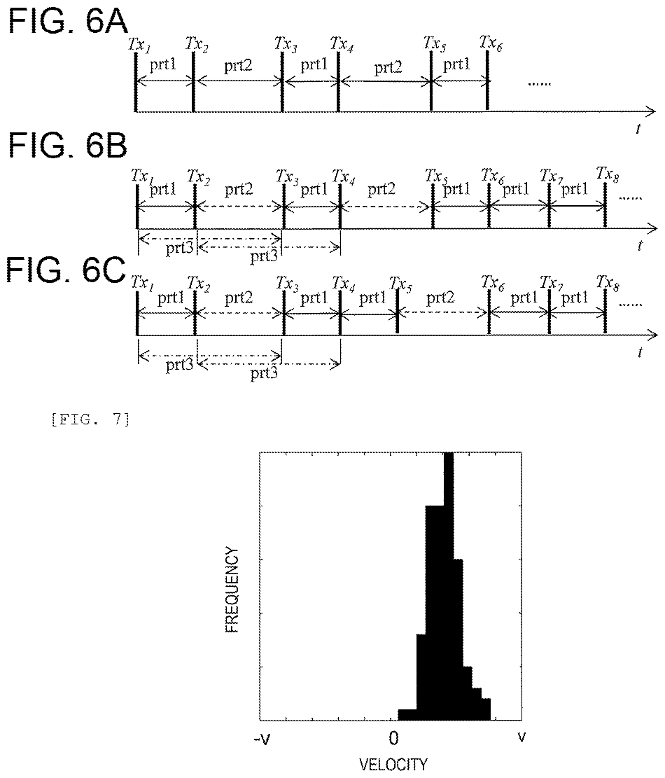

As a pulse sequence for aliasing avoidance, a transmission and reception sequence of a known uneven interval transmission color Doppler method may be used, or a method proposed by the present applicant (Japanese Patent Application No. 2018-40908, referred to as a prior application) may be used. In a known method, transmission and reception is performed with two or more different PRTs, and the velocity causing no aliasing is estimated by using a ratio of the PRTs. For example, as shown in FIG. 6(a), transmission is performed with the PTRs being alternately different (uneven interval transmission), and a signal set of prt1 and a signal set of prt2 are received for the PRTs. In the method described in the prior application, a transmission and reception sequence, as shown in FIG. 6(b), in which one of prt 1 and prt 2 (prt 1 in FIG. 6(b)) is repeated after prt 1 and prt 2 are alternately repeated, or a transmission and reception sequence, as shown in FIG. 6(c), in which prt 1 and prt 2 are alternately repeated in accordance with a predetermined rule is adopted. Accordingly, in estimating of the blood flow velocity, not only the signal set of prt 1 and the signal set of prt 2 are used, but also a signal set of prt 3 (=prt 1+prt 2), which is a third PRT, is used, and a decrease in frame rate accompanying usage of a plurality of types of PRTs is prevented.

In any of the methods, prt 1 and prt i (i=integer of 2 or more) are determined by satisfying the following relationship.

[Formula 1]

prfi=(pᵢ/qᵢ)×prf1 (1)

In the formula, pᵢ and qᵢ are integers in a nondivisible relationship, and are different from each other depending on “i”.

The transmission and reception of the ultrasonic wave in accordance with the sequence is performed on abeam line determined by the cursor 401 or on a plurality of beam lines including the vicinity thereof and a reflected wave from a sample position designated by the cursor 401 is sampled as a reception signal. Such transmission and reception is repeatedly performed, and a plurality of pieces of frame data is acquired.

The data distribution unit 81, for each piece of the frame data, divides reception signals obtained with a sequence using a plurality of PRTs into a signal set for each PRT (for example, a signal set of prt 1 and a signal set of prt 2), and transfers the reception signals to the measurement condition calculation unit 85 (blood flow velocity estimation unit 86) (S342). A main function of the data distribution unit 81 is to distribute signals corresponding to the measurement mode, that is, for example, to distribute signals received in the B-mode measurement to the tomographic image calculation unit 82, or to distribute signals received in the color Doppler measurement to the color Doppler calculation unit 83. In the present embodiment, when the transmission and reception for estimating the velocity causing no aliasing is further performed in the Doppler measurement as described above, a signal set for each PRT is distributed. However, this function may be provided separately from the data distribution unit 81, for example, upstream of the measurement condition calculation unit 85.

The blood flow velocity estimation unit 86 estimates the blood flow velocity by using data of the signal sets of the different PRTs (S343). The estimation of the blood flow velocity can be performed in accordance with a known method known as the uneven interval transmission color Doppler method. That is, when a blood flow velocity having aliasing obtained based on the PRTs (prt 1, prt i) is assumed to be V_{Di}, the Nyquist velocity is assumed to be V_{Ni}, and the number of times of aliasing is assumed to be n_{Ni}, the velocity V_{D} causing no aliasing to be estimated can be expressed by Formula (2).

[Formula 2]

V_{D}=V_{Di}+2n_{Ni}V_{Ni} (2)

Here, the Nyquist velocity is V_{N}=(PRF·C)/4f₀ (PRF is the pulse repetition frequency and the reciprocal of PRT; C is an ultrasonic velocity; and f₀ is a transmission frequency of an ultrasonic wave).

The number of times of aliasing satisfies a relationship of Formula (3), based on Formula (1).

[Formula 3]

V_{N1}=(pᵢ/qᵢ)×V_{Ni} (3)

Therefore, by solving the following Formula (4) derived from Formulas (2) and (3) using the following constraint conditions (Formulas (5) and (6)), the number of times of aliasing n_{N1} and n_{Ni} can be estimated.

[Formula 4]

nint[qᵢ×{(V_{Di}−V_{D1})/2V_{N1}}]=n_{N1}qᵢ−n_{Ni}pᵢ (4)

In Formula (4), “nint” is a conversion to an integer type.

[Formula 5]

|n_{N1}qᵢ−n_{Ni}pᵢ|≤(½)×(pᵢ+qᵢ) (5)

[Formula 6]

|n_{Ni}|≤ceiling{(qᵢ−1)/2} (6)

Since the velocity causing no aliasing is calculated for each PRT, the blood flow velocity estimation unit 86 obtains an average thereof as the velocity causing no aliasing. By performing the above calculations for each piece of the frame data, the velocity causing no aliasing is obtained for each piece of the frame data. Information about the velocity causing no aliasing for each piece of the frame data is stored in a memory for a predetermined period (S344). The blood flow velocity generally changes in accordance with a cardiac cycle. Therefore, storage of the blood flow velocity data is preferably performed over at least one cardiac cycle (about 1 second).

When the data storage for the predetermined period ends, the histogram generation unit 87 generates a blood flow distribution (histogram) of the blood flow velocities acquired during the predetermined period (for example, 1 second). In the histogram of the blood flow distribution, as in an example shown in FIG. 7, velocities at a target cursor and at the vicinity thereof are plotted according to the frequency. At this time, threshold processing (for example, processing of removing a lower limit of the minimum blood flow velocity as a threshold) is performed (S345), and a value that is obviously not contained in the blood flow velocities is removed from the blood flow velocity data. Meanwhile, when a position of the cursor 401 is changed within one frame as shown in FIG. 4(b) during the predetermined period, the position after change is taken as a target in the next frame and the above steps S341 to S344 are repeated. In the example shown in FIG. 4(b), since the cursor is changed from the scan line x to a scan line y and the sample window is changed from the samples e-f to samples g-h, the transmission and reception for estimating the velocity causing no aliasing is performed and the velocity causing no aliasing is estimated with this position as a target. The cursor is transmitted and received for the speed estimation without folding, and the aliasing velocity is estimated. If the position of the cursor 401 is not changed during the predetermined period (S346), information about the blood flow velocity during the predetermined time, for example, a time corresponding to one cardiac cycle, is finally obtained.

The measurement condition calculation unit 85 calculates spectral Doppler measurement conditions (for example, the velocity range and the baseline) by using the blood flow velocity information obtained in this way (S347). That is, the measurement condition calculation unit 85 determines a minimum velocity and a maximum velocity based on the histogram generated by the histogram generation unit 87, and with respect to a width (a difference between the minimum velocity and the maximum velocity), sets an appropriate range (for example, 120%) including the width as the velocity range. The baseline is set in a position where the maximum velocity does not alias, based on the histogram.

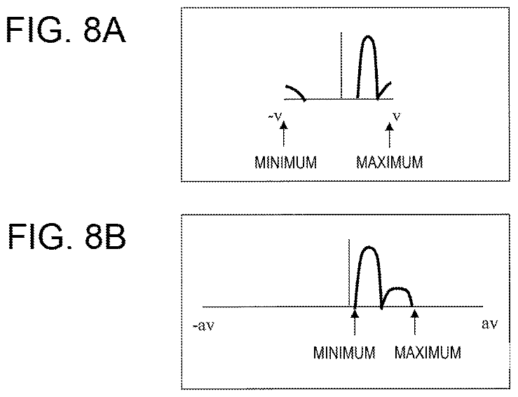

A case where the maximum velocity and the maximum velocity are determined by using the histogram is shown in FIG. 8. FIG. 8(a) shows a blood flow distribution of a case where the calculated blood flow velocity has aliasing (before aliasing correction), and a horizontal axis ±V shows a color Doppler measurement range. In the case where there is aliasing, since high velocity components are partially aliased in a minus direction, an accurate minimum blood flow velocity and maximum blood flow velocity cannot be obtained. In contrast, in a case where the velocity range is broadened and aliasing is corrected, since a distribution of velocity causing no aliasing is obtained and the minimum blood flow velocity and the maximum blood flow velocity can be detected accurately, an appropriate velocity range and baseline can be set. The “a” of ±aV on the horizontal axis in FIG. 8(b) is a speed range broadening width after the aliasing correction.

The measurement condition calculation unit 85 sets the calculated velocity range and baseline as initial measurement conditions for the following spectral Doppler measurement. The above steps S341 to S347 are performed during the intermediate period of transitioning from color Doppler to spectral Doppler, that is, from when the user sets the spectral Doppler measurement position with the cursor 401 for position designation to when the transmission and reception of the pulse for spectral Doppler is performed.

When an instruction to start spectral Doppler is sent out via the input unit 30, the spectral Doppler control unit 72 transmits and receives ultrasonic pulses under the set measurement condition (the velocity range and the baseline), and starts the measurement (FIG. 3: S35).

In the spectral Doppler measurement, an ultrasonic wave is transmitted to the measurement position (ultrasonic beam direction) designated by the cursor 401 in step S33, and a reflected wave from the sample position designated by the cursor 401 is received. Frame data of the reception signal is transferred to the spectral Doppler calculation unit 84 via the data distribution unit 81, and here frequency analysis is sequentially performed to generate a velocity spectrum. The velocity spectrum is converted to a display image by the display image generation unit 90 and displayed on the display unit 40.

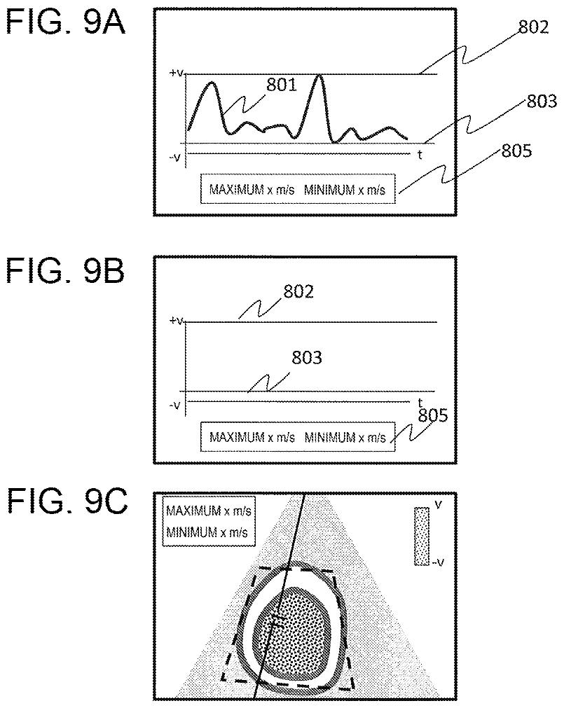

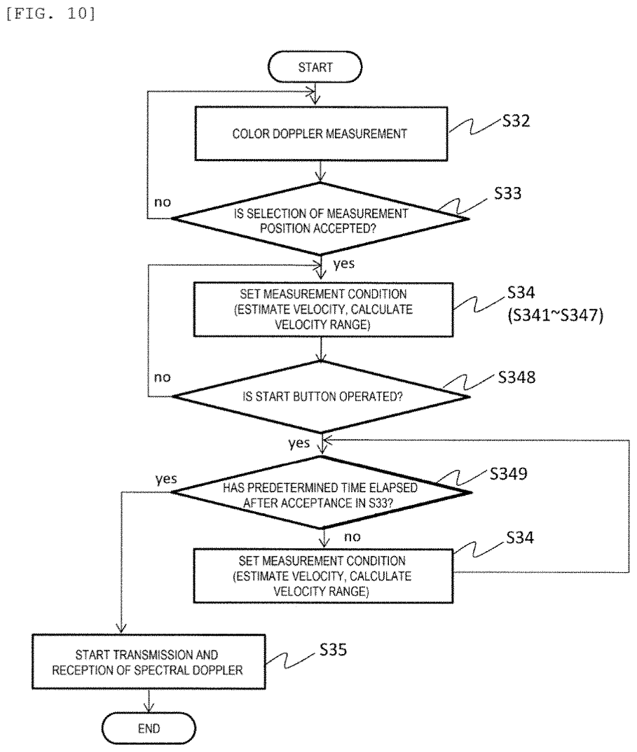

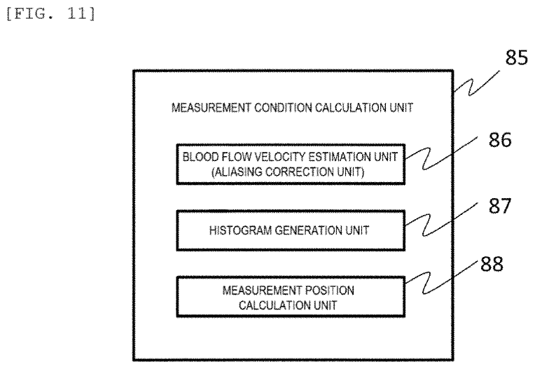

Here, in a case where the spectral Doppler measurement is pulse Doppler in which an ultrasonic wave is transmitted at a predetermined PRF, a maximum detection frequency depends on the PRF, and a maximum detection velocity determined by the maximum detection frequency is also limited by the PRF; in the measurement condition set initially, the PRF and the like are adjusted such that the velocity range (−V to +V) determined by the maximum detection velocity includes the width of the maximum blood flow velocity and the minimum blood flow velocity that are estimated by the blood flow velocity estimation unit 86, and the baseline is set in a position where the maximum blood flow velocity does not aliase. Therefore, for example, as shown in FIG. 9(a), a blood flow spectrum 801 is displayed in an appropriate range on a velocity display screen.

In the illustrated example, further, a maximum blood flow velocity 802 and a minimum blood flow velocity 803, which are estimated by the blood flow velocity estimation unit 86, are indicated by lines on the spectrum, and a display block 805 indicating values thereof is displayed. Since an appropriate velocity range and baseline is set at the start of the spectral Doppler measurement, an appropriate display can be implemented without adjustment by the user at the same time as the display of blood flow spectrum is started.

Before the spectrum that is a spectral Doppler measurement result is displayed, the maximum blood flow velocity 802, the minimum blood flow velocity 803 and the blood flow velocity display block 805 may be displayed on a spectrum display screen, as shown in FIG. 9(b). Alternatively, as shown in FIG. 9(c), the blood flow velocity display block 805 may be displayed on a display screen before the spectral Doppler measurement, for example, a display screen during color Doppler (intermediate period). By performing such displays, the user can check the propriety of the measurement position serving as a spectral Doppler measurement target, and determine the necessity of the spectral Doppler measurement in some cases. That is, if an object of the spectral Doppler measurement is only to obtain information about the maximum blood flow velocity (peak velocity), the measurement can be stopped in this state.

According to the present embodiment, the blood flow velocity causing no aliasing is estimated during the color Doppler measurement that is performed before spectral Doppler, and the spectral Doppler measurement conditions (the velocity range and the baseline) are calculated based on the blood flow velocity causing no aliasing and sets the spectral Doppler measurement conditions as initial conditions, so that adjustment by the user is not required and the measurement of the appropriate velocity range and the spectrum display can be performed at the same time as spectral Doppler is started.

According to the present embodiment, since it is ensured that the blood flow velocity for calculating the measurement conditions is the velocity causing no aliasing, the minimum and maximum blood flow velocities can be determined at the time of calculating the measurement conditions based on the accurate histogram.

### <First Modification>

In a first embodiment, it is disclosed that, in the intermediate period of transitioning from the color Doppler measurement to the spectral Doppler measurement, the transmission and reception is performed in the sequence for estimating the velocity causing no aliasing and measurement condition calculation is performed, but it is an extremely short time from when the cursor operation for selecting the spectral Doppler measurement position is performed during the color Doppler measurement to when the spectral Doppler start button is operated, which may be shorter than the performing time of the transmission and reception for aliasing avoidance (less than about one second). In this case, when the transmission and reception of ultrasonic pulses for spectral Doppler is started with, for example, a velocity range or the like that is set by default, a result of the measurement condition calculation unit 85 is not reflected.

In the present modification, the transmission control unit 70 restricts an operation of a start button from when a measurement position is selected through a cursor operation, until a predetermined time, for example, one second elapses, or provides a delay time from the operation of the start button to the transmission of the pulse for spectrum Doppler. Accordingly, it is ensured that the measurement conditions calculated by the measurement condition calculation unit 85 are the spectral Doppler initial conditions. A procedure of the transmission control unit 70 performing such restriction is shown in FIG. 10. In FIG. 10, processes same as those in FIGS. 3 and 5 are denoted by the same reference numerals and repetitive description thereof will be omitted. Further, in FIG. 10, a B-mode measurement step (FIG. 3: S31), which is a premise of color Doppler, is not shown.

When a selection of a measurement position is accepted (S33) during color Doppler measurement (S32), the measurement position or a narrow region including the measurement position is taken as a target, and estimation of a velocity causing no aliasing, and calculation of a velocity range and the like based on the velocity causing no aliasing is started (S34). Calculation processing of the velocity range and the like is the same as the flow shown in FIG. 5. As described above, the processing is performed, for example, over one cardiac cycle. When the start button for starting the transmission and reception in spectral Doppler is operated by a user during the processing (S348), it is determined whether the data storage over one cardiac cycle and measurement condition setting using the stored data is completed (S349), and if not completed, the transmission and reception is started after completion (S35). The determination of whether the measurement condition setting is completed may be performed based on elapsed time from when the selection of the measurement position is received, as shown in the figure, or the completion may be at a time point when a default measurement condition is updated after the spectrum Doppler control unit 72 receives the measurement conditions from the measurement condition calculation unit 85.

According to the first modification, even in a case where an intermediate period is extremely short and less than one second, the velocity causing no aliasing calculated based on reception signals acquired over one cardiac cycle can be reliably used, and setting of an accurate velocity range can be ensured.

In the flow of FIG. 10, the start of the transmission and reception is delayed by a control signal, but the control may be performed by electrically or mechanically locking the start button for a time corresponding to the delay time.

### <Second Modification>

In the first embodiment, in order to acquire the blood flow velocity causing no aliasing, the transmission and reception sequence for aliasing avoidance is used, and the blood flow velocity causing no aliasing is calculated through calculation of reception signals having different PRTs, but the method of acquiring the blood flow velocity causing no aliasing is not limited thereto, and a known method of correcting aliasing can be adopted. In this modification, the blood flow velocity estimation unit 86 in FIG. 2 functions as an aliasing correction unit.

Specifically, the following method can be adopted as an aliasing correction method.

Cross-correlation method: a method in which movements of one or more wavelengths of a received RF signal is obtained using a cross-correlation method and thereafter a velocity causing no aliasing is obtained by adding phase information obtained using an autocorrelation method thereto (for example, a method disclosed in non-Patent Literature 2: Lai X, et al, [An Extended Autocorrelation Method for Estimation of Blood Velocity], IEEE TRANSACTION On ULTRASONICS, FERROELECTRICS, AND FREQUENCY CONTROL, VOL. 44, No. 6, 1997).

Block matching method (template matching method): with respect to front frame data and rear frame data, a pair of corresponding points is obtained, and a small region including points around the corresponding points is taken as one unit and a corresponding relationship between frames is obtained. A velocity is obtained based on an amount of movement between a front frame and a rear frame of the corresponding points. As a criterion for obtaining the corresponding relationship, a sum of absolute values of differences (SAD), a sum of squares of differences (SDD), normalized cross-correlation, and the like are used.

In these modifications, it is not required to perform the transmission and reception sequence for aliasing avoidance as shown in FIG. 5, the transmission and reception can be performed under the same condition as that of color Doppler after the spectral Doppler measurement position is selected, and the blood flow velocity causing no aliasing can be calculated by using the received RF signal or frame data. The calculation (aliasing correction) of the blood flow velocity causing no aliasing according to the present modification can be implemented by changing an algorithm to be executed by the blood flow velocity estimation unit 86, and other device configurations and measurement procedures are similar to those of the first embodiment.

### Second Embodiment

In the first embodiment, the measurement position of the spectral Doppler measurement is selected by the user operating the cursor. In the present embodiment, the measurement position of the spectral Doppler measurement is automatically calculated by using information obtained in the color Doppler measurement.

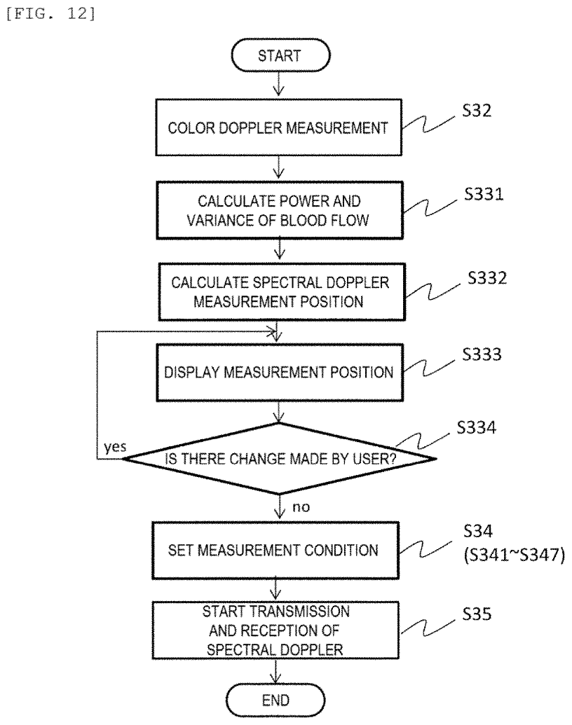

As shown in FIG. 11, the measurement condition calculation unit 85 of the present embodiment includes a measurement position calculation unit 88 in addition to the blood flow velocity estimation unit 86. The color Doppler calculation unit 83 uses a blood flow velocity for each sample of a color Doppler measurement region to calculate power and variance of a blood flow. The measurement position calculation unit 88 determines a measurement position, by using at least one of the blood flow velocity, the power and the variance that are calculated by the color Doppler calculation unit 83.

FIG. 12 illustrates a processing flow of the present embodiment. Steps in FIG. 12 for performing the same processing as steps in FIGS. 3 and 5, for performing the processing of the first embodiment, are denoted by the same reference numerals, and the repetitive description thereof will be omitted. Further, in FIG. 12, a B-mode measurement step (FIG. 3: S31), which is a premise of color Doppler, is not shown.

Also in the present embodiment, the spectral Doppler measurement (S35) is performed following the color Doppler measurement (S32) in a Doppler mode, and initial measurement conditions of the spectral Doppler measurement is calculated and set during an intermediate period of transitioning from the color Doppler measurement to the spectral Doppler measurement, which are similar to those in the first embodiment.

The color Doppler calculation unit 83 calculates the velocity, the power and the variance of the blood flow by using RF signals obtained in the color Doppler measurement (S331). A velocity Vel, signal power Pow, and variance Var at a certain point can be obtained with the following Formulas (7) to (9), and are calculated for each sample volume (measurement point).

$$\begin{matrix}Vel = {\sum }_{N} {E}_{N} * \overline{{E}_{N - 1}} & [ Formula 7 ] \\ Pow = {\sum }_{N} {E}_{N} * \overline{{E}_{N}} & [ Formula 8 ] \\ Var = 1 - \frac{\vert {\sum }_{N} {E}_{N} * \overline{{E}_{N - 1}} \vert}{Pow} & [ Formula 9 ]\end{matrix}$$

In the Formulas, E is an IQ signal after quadrature detection, and N is a data set number (the same applies hereinafter).

In general, in spectral Doppler, a position with the maximum blood flow velocity or power, or a position with high variance is taken as a measurement target. Therefore, the measurement position calculation unit 88 automatically sets, as a measurement position, a measurement point with a maximum value of a parameter among measurement points, with respect to predetermined parameters (blood flow velocity, power, variance) (S332). One or a plurality of measurement positions may be set.

The measurement condition calculation unit 85 performs processing for estimating a velocity causing no aliasing as in the first embodiment, with respect to the set measurement position (S34). That is, for example, the velocity causing no aliasing is estimated by (blood flow velocity estimation unit 86) using reception signals obtained by performing uneven interval transmission, the minimum blood flow velocity and the maximum blood flow velocity are obtained based on a histogram of the velocity causing no aliasing which is obtained during a predetermined time (about one second), and a velocity range and a baseline in spectral Doppler, are calculated. Next, the calculated velocity range and baseline are set as initial measurement conditions of spectral Doppler.

The measurement position calculation unit 88 may send position information about the automatically set measurement position to the display image generation unit 90 to display the position information on a color Doppler display screen (S333). Accordingly, a user can check the automatically set measurement position. At this time, a change of the measurement position by the user may be accepted, and if the user changes the measurement position, the change of the measurement position is accepted as in the first embodiment (S334).

When the measurement position calculation unit 88 determines the measurement position, the transmission control unit 70, at that time, executes step S34 (S341 to S347 in FIG. 4) of the first embodiment, and sets the initial measurement conditions before the start of spectral Doppler. In a case where the measurement position is displayed on the display screen and a change is made to the measurement position by the user, the step S34 is executed as in the case where the measurement position is selected through the cursor operation by the user in the first embodiment.

Also in the present embodiment, by using the estimated velocity causing no aliasing, an accurate velocity range and baseline can be set, and these measurement conditions can be set at the start of spectral Doppler without intervention of the user. Further, in the present embodiment, the measurement position is also automatically set, so that waiting time of the user can be further shortened, and the convenience can be enhanced.

### <Third Modification>

In the first embodiment, the transmission and reception is performed in a sequence (transmission and reception sequence for aliasing avoidance) for acquiring signals necessary for estimating the blood flow velocity causing no aliasing in a period between color Doppler and spectral Doppler, that is, in the intermediate period, but the transmission and reception may be performed in such a sequence during the transmission and reception of color Doppler.

Also in the first embodiment, since the transmission and reception sequence of color Doppler may be continued after the transmission and reception is performed in the sequence for aliasing avoidance and before the start of the transmission and reception of spectrum Doppler, the execution of the transmission and reception in the sequence for aliasing avoidance, and subsequent measurement condition calculation can be referred to as processing performed during the color Doppler measurement. This third modification is characterized in that the transmission and reception is performed in the sequence for aliasing avoidance without waiting for the user to select a measurement position.

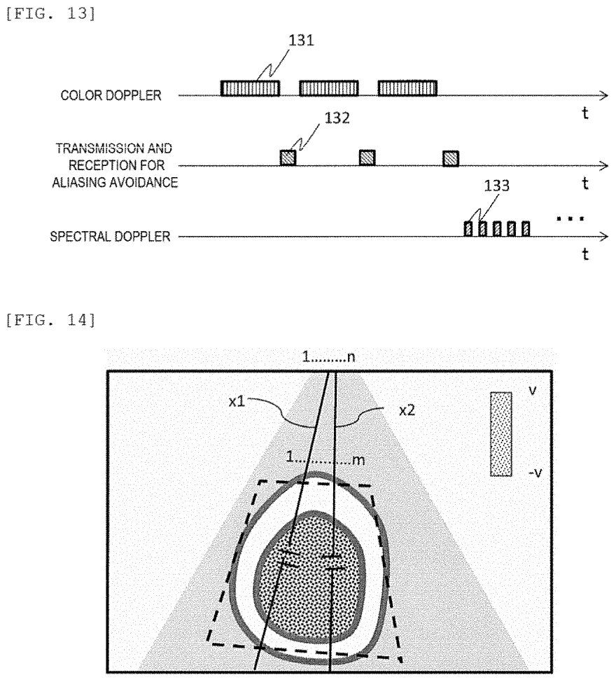

FIG. 13 shows an example of transmission and reception sequence of the present modification. In FIG. 13, in a transmission and reception sequence 131 of color Doppler, one square indicates transmission and reception of one or more pieces of frame data. In the present modification, as illustrated, every time one piece of frame data, for example, is acquired in color Doppler, transmission and reception is performed in a transmission and reception sequence 132 (any one of FIG. 6) for aliasing avoidance for a predetermined period (about one second), and a velocity causing no aliasing (minimum blood flow velocity, maximum blood flow velocity) is acquired. The transmission and reception is performed in the transmission and reception sequence 132 during the transmission and reception sequence 131 of color Doppler before a spectral Doppler measurement position is determined, and an ultrasonic beam direction and a sample position are not determined and will be set with another method. For example, as shown in FIG. 14, scan lines x1 and x2, which are one or more measurement position candidates, and a depth are automatically or manually set in advance within a color Doppler scan range, and the scan lines or a plurality of scan lines including the scan lines are taken as measurement targets of the transmission and reception sequence 132. When there are a plurality of candidates, the velocity causing no aliasing is calculated for each candidate. When the measurement position calculation unit 88 automatically calculates the measurement position by using the method of the second embodiment, the transmission and reception is performed in the transmission and reception sequence 132 at a measurement position calculated by the measurement position calculation unit 88 for each color Doppler frame.

When there are a plurality of measurement positions, the measurement condition calculation unit 85 stores, for each measurement position, blood flow information (minimum blood flow velocity and maximum blood flow velocity that are obtained based on the velocity causing no aliasing) acquired through the transmission and reception sequence 132 in a memory.

During transitioning from color Doppler to spectral Doppler, that is, when an instruction to change a measurement mode to spectral Doppler is input during color Doppler measurement, the transmission control unit 70 displays the cursor 401 (FIG. 4) for selecting a spectral Doppler measurement position on the display unit 40. A measurement position that is the measurement target of the transmission and reception sequence 132 is displayed as an initial position of the cursor 401. When there are a plurality of measurement position candidates, the plurality of measurement position candidates may be displayed.

When a measurement position selected through a cursor operation by a user is the same with or in the vicinity of a position of the measurement position candidate displayed as the initial position or any one of the plurality of measurement position candidates, since the minimum and maximum blood flow velocities are regarded as substantially the same, the spectral Doppler measurement (transmission and reception sequence 133) can be started at the measurement position selected by the user, in a velocity range and baseline that are determined based on stored blood flow information about the measurement position candidates, without performing the measurement condition setting processing (S34 in FIG. 5) again for the measurement position selected by the user.

According to the present modification, even before the spectral Doppler measurement position is determined, the spectral Doppler measurement conditions can be preliminarily determined during execution of the color Doppler measurement in the transmission and reception sequence. Accordingly, the convenience for the user can be further enhanced while the setting of an accurate velocity range and the like can be ensured in spectral Doppler.

Although the embodiments of the ultrasonic imaging device and the control method thereof of the invention have been described above, the invention is not limited to these embodiments. Known elements may be added or a part of the elements may be omitted. The embodiments and modifications can be appropriately combined as long as there is no technical contradiction, and such a combination is also contained in an embodiment of the invention.

### REFERENCE SIGN LIST

10: main body, 20: ultrasonic probe, 30: input unit, 40: display unit, 60: transmission and reception circuit, 70: transmission control unit (control unit), 71: color Doppler control unit, 72: spectral Doppler control unit, 80: signal processing unit (calculation unit), 81: data distribution unit, 82: tomographic image calculation unit, 83: color Doppler calculation unit, 84: spectral Doppler calculation unit, 85: measurement condition calculation unit, 86: blood flow velocity estimation unit, 87: histogram generation unit, 88: measurement position calculation unit, 90: display image generation unit

1. An ultrasonic imaging device, comprising:
a transmission and reception circuit that transmits and receives an ultrasonic signal via an ultrasonic probe;
a calculation unit that performs Doppler calculation using an ultrasonic signal received by the transmission and reception circuit; and
a control unit that controls an operation of the transmission and reception circuit, and performs a first blood flow measurement for acquiring a two-dimensional distribution of blood flow information, and a second blood flow measurement for acquiring a spectrum of a blood flow velocity, wherein
the calculation unit includes a blood flow velocity estimation unit that estimates a blood flow velocity causing no aliasing by using an ultrasonic signal acquired before transmission and reception of a ultrasonic wave in the second blood flow measurement is started, and a measurement condition calculation unit that calculates a measurement condition of the second blood flow measurement by using the blood flow velocity causing no aliasing.

2. The ultrasonic imaging device according to claim 1, wherein
the control unit starts the second blood flow measurement under the measurement condition calculated by the measurement condition calculation unit.

3. The ultrasonic imaging device according to claim 1, wherein
the control unit performs a measurement that uses an ultrasonic signal of a plurality of pulse repetition frequencies during the first blood flow measurement, and
the blood flow velocity estimation unit estimates the blood flow velocity causing no aliasing by using the plurality of pulse repetition frequencies and an ultrasonic signal acquired in a measurement that uses the plurality of pulse repetition frequencies.

4. The ultrasonic imaging device according to claim 3, wherein
the control unit executes the measurement that uses an ultrasonic signal of a plurality of pulse repetition frequencies for a predetermined period.

5. The ultrasonic imaging device according to claim 1, wherein
the blood flow velocity estimation unit estimates the blood flow velocity causing no aliasing based on time-series ultrasonic signals acquired in the first blood flow measurement, by using any one of a cross-correlation method and a block matching method.

6. The ultrasonic imaging device according to claim 1, wherein
the blood flow velocity estimation unit creates a histogram for a blood flow velocity that is estimated based on ultrasonic signals acquired in a predetermined period, and estimates a maximum blood flow velocity and a minimum blood flow velocity based on the histogram.

7. The ultrasonic imaging device according to claim 1, wherein
the measurement condition calculation unit calculates a measurement condition including at least one of a velocity range and a baseline by using a maximum blood flow velocity and a minimum blood flow velocity that are estimated by the blood flow velocity estimation unit.

8. The ultrasonic imaging device according to claim 1, further comprising:
an accepting unit that accepts a measurement position of the second blood flow measurement by a user, wherein
when the accepting unit accepts the measurement position in the first blood flow measurement, the blood flow velocity estimation unit estimates the blood flow velocity causing no aliasing by using an ultrasonic signal received from the measurement position.

9. The ultrasonic imaging device according to claim 8, wherein
the blood flow velocity estimation unit calculates a maximum velocity and a minimum velocity based on the blood flow velocity causing no aliasing, and the control unit causes a display device to display the measurement position accepted by the accepting unit as well as the maximum velocity and the minimum velocity.

10. The ultrasonic imaging device according to claim 8, wherein
when the measurement position accepted by the accepting unit is changed, the blood flow velocity estimation unit discards a blood flow velocity estimated before the change, and estimates another blood flow velocity by using an ultrasonic signal received from a measurement position after the change.

11. The ultrasonic imaging device according to claim 1, further comprising:
a display unit that displays a spectrum acquired in the second blood flow measurement, wherein
at the start of the second blood flow measurement, the control unit causes the display unit to display information about the blood flow velocity estimated by the blood flow velocity estimation unit and/or the measurement condition calculated by the measurement condition calculation unit.

12. The ultrasonic imaging device according to claim 1, wherein
the calculation unit further includes a measurement position calculation unit that calculates a measurement position of the second blood flow measurement by using blood flow information obtained in the first blood flow measurement.

13. A control method of an ultrasonic imaging device including a transmission and reception circuit that transmits and receives an ultrasonic signal via an ultrasonic probe and a calculation unit that performs Doppler calculation by using an ultrasonic signal received by the transmission and reception circuit, the control method comprising:
a step of causing the transmission and reception circuit to perform a first blood flow measurement for acquiring a two-dimensional distribution of blood flow information and a second blood flow measurement for acquiring a spectrum of a blood flow velocity; and
a step of causing the calculation unit to perform calculation of estimating a blood flow velocity causing no aliasing by using an ultrasonic signal acquired during the first blood flow measurement, and to perform calculation of calculating a measurement condition of the second blood flow measurement by using the blood flow velocity causing no aliasing, wherein
transmission and reception of an ultrasonic signal of the second blood flow measurement is started under the measurement condition calculated by the calculation unit.

14. The control method of an ultrasonic imaging device according to claim 13, further comprising:
a step of accepting a measurement position of the second blood flow measurement by a user during the first blood flow measurement, wherein
the calculation of estimating a blood flow velocity is performed by using an ultrasonic signal received from an accepted measurement position.

15. The control method of an ultrasonic imaging device according to claim 13, wherein
the calculation of estimating a blood flow velocity includes calculation of estimating a maximum blood flow velocity and a minimum blood flow velocity based on ultrasonic signals acquired in a predetermined period in the first blood flow measurement, and
at the start of the second blood flow measurement, the maximum blood flow velocity and the minimum blood flow velocity are displayed on a display screen that displays a result of the second blood flow measurement.
